(19) United States
(12) Patent Application Publication
(10) Pub. No.: US 2013/0098844 A1
(43) Pub. Date: Apr. 25, 2013

(54) Method and Arrangement for a Water Treatment Control

(71) Applicant: Siemens Aktiengesellschaft, Munchen (DE)

(72) Inventors: Dieter Forstmeier, Burgau (DE); Chona Dela Cruz Garcia, Berlin (DE); Cosima Sichel, Ulm (DE); Johannes Stein, Ulm (DE)

(73) Assignee: SIEMENS AKTIENGESELLSCHAFT, Munchen (DE)

(21) Appl. No.: 13/655,790

(22) Filed: Oct. 19, 2012

(30) Foreign Application Priority Data
Oct. 19, 2011 (EP) 11185769.4

(51) Int. Cl. G01N 33/18; C02F 1/76

(52) U.S. Cl. 210/739; 210/96.1; 73/61.43; 73/19.1

(57) ABSTRACT

The present invention relates to water treatment control (1, 100) of water (3) to be treated containing a chlorine disinfectant (5) stabilized by a chlorine stabilizer (6). A concentration of said chlorine disinfectant (5) contained in said water (3) will be measured (110) once before irradiating (120) said water (3) with an UV irradiation (initial chlorine concentration). After irradiating (120) said water (3) with said UV irradiation said concentration of said chlorine disinfectant 5 contained in said water 3 will be measured (130) twice (remaining chlorine concentration). A concentration of said chlorine stabilizer 6 added to said water will be determined (140) using said initial chlorine concentration and said remaining chlorine concentration for said water treatment control (1, 100).

### CROSS-REFERENCE TO RELATED APPLICATIONS

This application claims priority to EP Patent Application No. 11185769.4 filed Oct. 19, 2011. The contents of which is incorporated herein by reference in its entirety.

### TECHNICAL FIELD

The present invention relates to water treatment control of water to be treated containing a chlorine disinfectant stabilized by a chlorine stabilizer.

### BACKGROUND

Common disinfection methods for water, for example swimming pool water, include a dosing of a disinfectant, commonly a chlorine disinfectant, such as chlorine/chlorine gas (Cl2), chlorine dioxide (ClO2), hypochloric acid (HOCl) or hypo chlorite (OCl—).

It is known that said chlorine disinfectant dosed to/contained in said water are very instable to UV irradiation—being degraded by said UV irradiation (photolytic chlorine degradation) (Mariko Tachikawa et al. “Effects of Isocyanuric Acid on the Monochlorodimedone Chlorinating rates with Free Chlorine and Ammonia Chloramine in Water”, Water Research, 36 (2002), pp. 2547-2554; J. Gardiner, “Choloisocyanurates in the Treatment of Swimming Pool Water”, Water Research Pergamon Press 1973, Vol. 7, pp. 823-833).

Especially by “open” water chlorine disinfection, particularly outdoor swimming pool water chlorine disinfection, said chlorine disinfectant dosed/contained in said “open” water, i.e. said outdoor swimming pool water, will strongly be degraded by sunlight (photolytic chlorine degradation).

An effect of photolytic chlorine degradation will increase for regions of high solar irradiance, for example southern European countries, but can also be relevant and/or significant—on sunny days—in other regions, such as northern European countries.

Photolytic chlorine degradation by water chlorine disinfection can be balanced by an increased chlorine disinfectant dosing, to maintain same, i.e. consistent effective, chlorine disinfectant concentration in said water to disinfect.

This effort will increase process costs while being unsatisfying for an operator/customer. Besides disinfecton by-products which have negative health effects on bathers can increase due to increased chlorine consumption.

Another option can be an adding of a chlorine disinfectant stabilizing compound (chlorine stabilizer) to said water, stabilizing said chlorine disinfectant, i.e. improving a UV stability of said chlorine disinfectant.

A most successful of this compound in use at present is isocyanuric acid, which with chlorine species, i.e. chlorine disinfectant, forms reversibly N-chlorinated chloroisocyanurates in aqueous solution, i.e. in said water, and stabilizing said chlorine disinfectant (Mariko Tachikawa et al. “Effects of Isocyanuric Acid on the Monochlorodimedone Chlorinating rates with Free Chlorine and Ammonia Chloramine in Water”, Water Research, 36 (2002), pp. 2547-2554; J. Gardiner, “Choloiso-cyanurates in the Treatment of Swimming Pool Water”, Water Research Pergamon Press 1973, Vol. 7, pp. 823-833; C. J. Downes et al., “Determination of Cyanuric Acid Levels in Swimming Pool Waters By u.v. Absorbance, HPLC and Melamine Cyanurate Precipitation”, Water Research Vol. 18, No. 3, pp. 277-280, 1984).

Chlorine disinfection of the (swimming pool) water can be conveniently carried out with chlorinated isocyanuric acid, for example solid chloroisocyanurate (tablets), usually a sodium or potassium salt of a dichloro compound, while said chlorinated isocyanurate species forming mon-, di- and tri-chlorinated species in a presence of said chlorine disinfectant dissolved in water, i.e. of free chlorine (J. Gardiner, “Choloisocyanurates in the Treatment of Swimming Pool Water”, Water Research Pergamon Press 1973, Vol. 7, pp. 823-833; C. J. Downes et al., “Determination of Cyanuric Acid Levels in Swimming Pool Waters By u.v. Absorbance, HPLC and Melamine Cyanurate Precipitation”, Water Research Vol. 18, No. 3, pp. 277-280, 1984).

In (swimming pool) water often chemical equilibrium were reported between said free chlorine and the chlorinated isocyanurate species.

In an absence of an adequate (online) monitoring of a chlorine disinfectant concentration and a concentration/level of said chlorine stabilizer, i.e. of said isocyanuric acid—in following also referred as isocyanic acid, while disinfecting said water, especially swimming pool water, with said chlorine disinfectant being stabilized with said chlorine stabilizer, said chlorine stabilizer could accumulate within said water, i.e. swimming pool water, endangering disinfection safety and water quality.

Known (concentration) measurement methods for said chlorine disinfectant and said isocyanuric/isocyanic acid are spectrometric methods (DPD, Melanin based CyA-Test). Only said chlorine disinfectant concentration can be as well monitored by online sensors.

Online measurements of said chlorine stabilizer concentration, especially of said isocyanic acid concentration, for water treatment applications have not yet been developed.

Such an online chlorine stabilizer measurement would not only allow to measure and/or monitor said chlorine stabilizer concentration, i.e. said isocyanic acid concentration, in water to be treated/disinfected, especially in swimming pool water.

Online chlorine stabilizer measurement also can facilitate an (online) water treatment/disinfection control—with substantial and extensive controlling functions/functionalities such as dosing water treatment components, as said chlorine stabilizer and said chlorine disinfectant,—for quality and cost optimized water, especially swimming pool water, treatment conditions.

Equipments for dosing as well as for a controlled dosing of chlorine disinfectant to water to be treated are known as well as equipments for irradiating water with UV, for example “Wallace & Tiernan®, Wasseraufbereitungs- and Desinfektionssysteme, Oktober 2010.

(Online) Sensors measuring chlorine disinfectant concentrations of chlorine disinfectant contained in water are known, for example a membrane sensor FC1 (Free Chlorine) or TC1 (Total Chlorine) as well as a bare electrode sensor Depolox5 of Wallace & Tiernan (Wallace & Tiernan, Siemens, Water Technologies, Produktinformation zu Membransensor FC1, TC1 and zu Depolox5 Sensor).

### SUMMARY

In one embodiment, a method for a water treatment control of water to be treated containing a chlorine disinfectant stabilized by a chlorine stabilizer comprises the following steps: measuring a concentration of said chlorine disinfectant contained in said water before irradiating said water with an UV irradiation (initial chlorine concentration), irradiating said water with said UV irradiation, measuring said concentration of said chlorine disinfectant contained in said water after said irradiating of said water (remaining chlorine concentration), and determining a concentration of said chlorine stabilizer using said initial chlorine concentration and said remaining chlorine concentration for said water treatment control.

In a further embodiment, said chlorine disinfectant is chlorine/chlorine gas, chlorine dioxide, hypochloric acid or hypo chlorite. In a further embodiment, said chlorine stabilizer is isocyanic acid. In a further embodiment, said water to be treated is a swimming pool water. In a further embodiment, a change between said initial and said remaining chlorine concentration is determined while determining said concentration of said chlorine stabilizer, especially with an increase or decrease in said change between said initial and said remaining chlorine concentration correlating with a decrease or increase of said concentration of said chlorine stabilizer. In a further embodiment, the method is used for an online measurement and/or online monitoring of said chlorine stabilizer with said initial and remaining chlorine concentration being online measured and/or said online measured initial/remaining chlorine concentration being online processed to determine said concentration of said chlorine stabilizer online. In a further embodiment, said water treatment control uses said online measured and/or online monitored chlorine stabilizer concentration.

In another embodiment, an arrangement for a water treatment control of water to be treated containing a chlorine disinfectant stabilized by a chlorine stabilizer comprises: a flow pass of said water flowing at said flow pass with an UV source arrangement arranged at said flow pass for irradiating said water with an UV irradiation, a first measuring means arranged at said flow pass upstream of said UV source arrangement for measuring a concentration of said chlorine disinfectant contained in said water before irradiating said water with said UV irradiation (initial chlorine concentration), a second measuring means arranged at said flow pass downstream of said UV source arrangement for measuring said concentration of said chlorine disinfectant contained in said water after said irradiating of said water (remaining chlorine concentration), and a controlling means for determining a concentration of said chlorine stabilizer using said initial chlorine concentration and said remaining chlorine concentration for said water treatment control.

In a further embodiment, said UV source arrangement comprises one or more tubular low pressure UV lamps, especially operating in a range of 20 Watt-100 Watt and/or with an irradiation dose of about 200 J/m²-4000 J/m². In a further embodiment, the arrangement for a water treatment control is arranged at a “by pass” of a main water circulation, especially of a main pool water circulation.

### BRIEF DESCRIPTION OF THE DRAWINGS

Example embodiments will be explained in more detail below with reference to figures, in which:

FIG. 1 is a schematic illustration of an online measurement system for isocyanic acid according to an example embodiment of the present disclosure, and

FIG. 2 is a schematic illustration of an online measurement process for isocyanic acid according to an example embodiment of the present disclosure.

### DETAILED DESCRIPTION

Some embodiments provide a method and an arrangement by which the above-mentioned shortcomings in water treatment control can be mitigated.

For example, some embodiments provide a method and an arrangement which facilitate an online monitoring and/or measurement of a chlorine stabilizer as well as an (online) water treatment/disinfection control.

Some embodiments provide a method and an arrangement for a water treatment control of water to be treated containing a chlorine disinfectant stabilized by a chlorine stabilizer.

The method may comprise the following steps:

A concentration of said chlorine disinfectant contained in said water will be measured once before irradiating said water with an UV irradiation (initial chlorine concentration).

After irradiating said water with said UV irradiation said concentration of said chlorine disinfectant contained in said water will be measured twice (remaining chlorine concentration).

A concentration of said chlorine stabilizer contained in said water will be determined using said initial chlorine concentration and said remaining chlorine concentration for said water treatment control.

The arrangement may comprise:
a flow pass of said water flowing at said flow pass with an UV source arrangement arranged at said flow pass for irradiating said water with an UV irradiation,
a first measuring means arranged at said flow pass upstream of said UV source arrangement for measuring a concentration of said chlorine disinfectant contained in said water before irradiating said water with said UV irradiation (initial chlorine concentration)
a second measuring means arranged at said flow pass downstream of said UV source arrangement for measuring said concentration of said chlorine disinfectant contained in said water after said irradiating of said water (remaining chlorine concentration),
a controlling means for determining a concentration of said chlorine stabilizer contained in said water using said initial chlorine concentration and said remaining chlorine concentration for said water treatment control.

In other words—certain embodiments relate to a measurement/determination of said chlorine stabilizer contained in said disinfected water with a measurement principle is based on the photolytic chlorine degradation.

The chlorine concentration of said water containing said chlorine disinfectant and said chlorine stabilizer, i.e. treated with said stabilized chlorine disinfectant, is measured in front of/before (initial chlorine concentration) and past/after (remaining chlorine concentration) said UV irradiation of said water—leading to a Δ in the chlorine concentration (Δchlorine).

The degradation of said chlorine disinfectant, i.e. said Δchlorine, correlates, for example is proportional, to said chlorine stabilizer concentration, as for example said isocyanic acid concentration.

Experiments have shown that a decrease in photolytic chlorine degradation—resulting in a decreased Δchlorine—is close to linear proportional to an increase in chlorine stabilizer concentration.

In other words—an increased concentration of said chlorine stabilizer contained in said water could be seen by decreased photolytic chlorine degradation, i.e. Δchlorine measured.

An exact UV/chlorine stabilizer dose response curve, for example an UV/isocyanic acid dose response curve, can be determined experimentally—and then used for determining said chlorine stabilizer concentration using said (measured) initial chlorine concentration and said (measured) remaining chlorine concentration, i.e. using said Δchlorine measured.

Hence, the concentration of said chlorine stabilizer is determined based on the change in free chlorine photolysis—easily measured by (online) chlorine sensors providing said chlorine stabilizer being online measured.

Online chlorine stabilizer measurement facilitate an (online) water treatment/disinfection control—with said substantial and extensive controlling functions/functionalities such as dosing water treatment components for quality and cost optimized water, especially swimming pool water, treatment conditions.

Therefore, certain embodiments provide a new, effective approach for efficient water treatment/disinfection control reaching a targeted water quality at a very economic, ecological and practical way.

According to one embodiment said chlorine disinfectant is chlorine/chlorine gas (Cl2), chlorine dioxide (ClO2) hypochloric acid (HOCl) or hypo chlorite (OCl—) as long as these chlorine disinfectants are most successful in use at present.

According to a further embodiment said chlorine stabilizer is isocyanic acid. But as well other cynic species and similar chemicals, improving said UV stability of chlorinated species, i.e. said chlorine disinfectant, could be used.

Said chlorine disinfectant and said chlorine stabilizer can be combined as chlorinated or non chlorinated isocyanic acid being added/dosed to said water to be treated/disinfected. Hence, stabilized chlorine disinfection of the (swimming pool) water can be conveniently carried out with said chlorinated isocyanuric acid, for example dosed/dosing solid chloroisocyanurate (tablets, grains, powder) or in liquid form to said (swimming pool) water.

According to one embodiment said UV source arrangement is one or more UV source/sources irradiating said water containing said chlorine disinfectant and chlorine stabilizer. Said UV source arrangement can have any shape and/or size, preferable hollowly coaxial or of cylindrical shape with the one or more UV source/sources inside and being flowed through by said water.

Said UV irradiation could be applied with an irradiation dose of about 200 J/m²-4000 J/m². Furthermore said UV irradiation could have a wavelength of about 100 nm-400 nm, especially having a wavelength of about 200 nm-400 nm, furthermore especially of about 240 nm-300 nm.

The UV source arrangement could be selected with the degradation in chlorine concentration can easily be detected but as well as regarding capital and operational cost of the arrangement.

In one embodiment said UV source arrangement comprises a polychromatic irradiator/medium pressure UV source. Medium pressure UV sources/lamps provide an expanded wavelength spectrum and could be constructed more compact.

Said UV source arrangement could also be a mono-chromatic irradiator/low pressure UV source, for example a low pressure amalgam UV lamp or a low pressure mercury UV lamp. Low pressure UV lamps are highly efficient while providing a small spectrum by a wavelength of about 257.3 nm, less energy input combined with less costs.

In one embodiment said UV source arrangement comprises one or more tubular low pressure UV lamps operating in a range of 20 Watt-100 Watt.

As well solar irradiance can be used as an UV source as well as excimer lights or UV-LEDs or even light sources emitting below a UV spectrum in visible light range (with lower photolysis yields).

Furthermore an UV sensor (or more)—for a low pressure UV source or a medium pressure UV source—and/or a UV filter (or more) could be used in combination with said UV irradiation provided by said UV source arrangement, e.g. low pressure UV source or medium pressure UV source, for controlling an irradiance of said UV irradiation.

A flow rate of said water—while being measured—and/or a reaction time, i.e. an irradiation time, could be kept constant as the photolytic chlorine degradation would depend on the irradiation (W) and the reaction time (T), i.e. on the dose (D) or fluence (F). It could—while not being kept constant—as well be measured and integrated as parameter into the overall measurement.

Said first and/or second measuring means for measuring said initial/remaining chlorine concentration may comprise online chlorine sensor(s), e.g., measuring free chlorine, i.e., free chlorine concentration.

Such a sensor, for example a membrane sensor FC1 or TC1 (Free Chlorine/Total Chlorine) or a bare electrode sensor Depolox5 of Wallace & Tiernan (Wallace & Tiernan, Siemens, Water Technologies, Produktinformation zu Membransensor FC1, TC1 and zu Depolox5 Sensor), is well known, long term stable while measuring and requires low maintenance costs.

But as well a sensor/sensors for total chlorine or combined chlorine species or similar devices/means could provide an adequate chlorine signal being used for determining said chlorine stabilizer concentration.

Sensor/measurement means signals and/or sensor/measurement means data according to said measured initial and/or remaining chlorine concentration(-s) could be processed online by said controlling means, for example a processing unit provided with measurement/controlling software, facilitating said determination of said chlorine stabilizer concentration for said water treatment control.

Said water treatment controlling could include a dosing and/or an adjustment of adding/dosing of said chlorine disinfectant and/or said chlorine stabilizer and/or said combined stabilized chlorine disinfectant facilitating disinfection as well as stabilisation of said chlorine disinfectant, such as chlorinated isocyanic acid (tablets), to said water.

Said water treatment controlling could also include controlling said irradiating/UV irradiation source as well as controlling said flow rate of said water to be treated/disinfected.

According to one embodiment said water to be disinfected flows at a flow rate typically for respective water treatment controlling measurement systems, for example at a flow rate of 1 L/h-50 L/h, especially at a, especially constant, flow rate of about 5 L/h-35 L/h. The flow rate can be controlled/monitored by use of a flow control.

According to one embodiment said water to be disinfected could be swimming pool water. But nevertheless said method and arrangement could be used for any disinfection application that need UV stabilization of said chlorine disinfectant, particularly whose free chlorine.

Said method and/or arrangement for said water treatment control could be applied to said water while said water flows in a main water circulation, for example a swimming pool water circulation, particularly that is treated by the UV device serving as well as said arrangement for the chlorine stabilizer determination, particularly for an isocyanuric acid measurement, as well as said water flows in a “by pass” flow being arranged in parallel to said main water circulation. As another option the water can be discharged after the measurement in by pass.

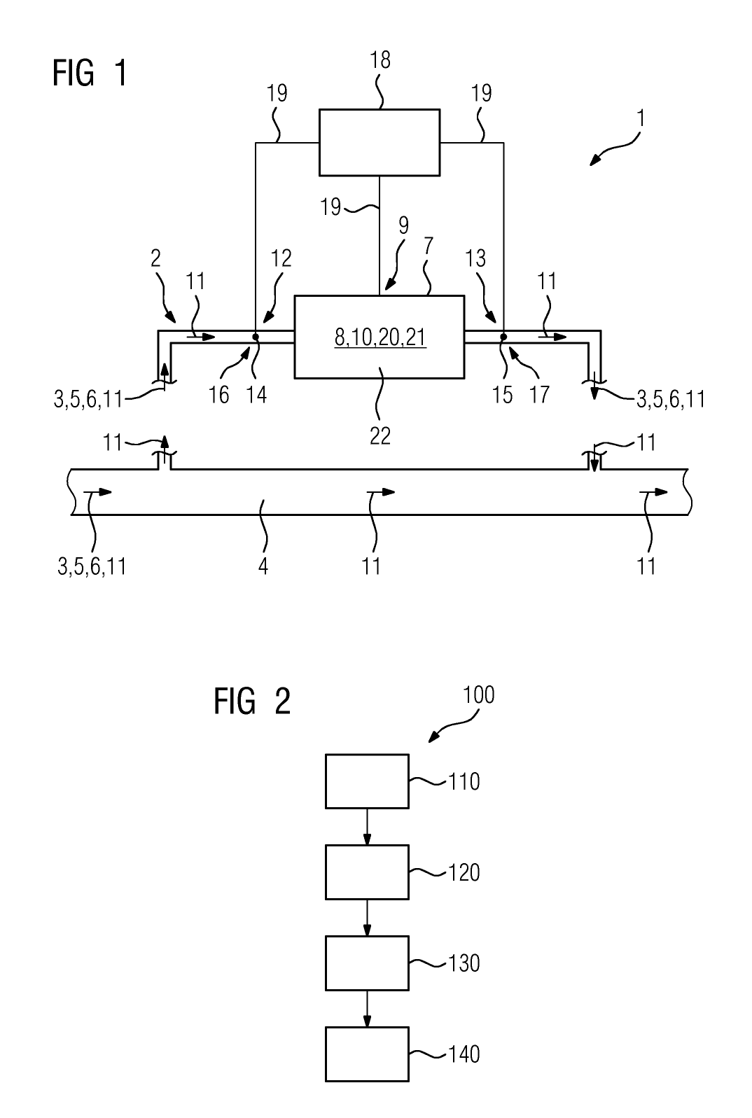

FIGS. 1 and 2 illustrate an example arrangement 1 and method 100, respectively, for a water treatment/chlorine disinfection control facilitating an online measurement of a chlorine stabilizer, i.e. of isocyanic acid.

The water treatment/chlorine disinfection control 1, 100 facilitating said online isocyanic acid measurement as schematically illustrated in FIG. 1 and FIG. 2 will be used for disinfecting swimming pool water 3 while being applied to a “by pass” 2 of a main water circulation of said swimming pool water circulating 4.

The swimming pool water 3, only referred as water 3, to be disinfected by a chlorine disinfectant 5, especially by chlorine 5, contains hazardous contaminants, especially residuals of pesticides, pharmaceuticals, drugs, hormones, personal care products—which can be eliminated by the water chlorine disinfection, i.e. by said chlorine 5.

As it is known that said chlorine disinfectant 5, as said chlorine 5, is very instable to UV irradiation and being degraded by said UV irradiation 7 (photolytic chlorine degradation 8) a chlorine disinfectant stabilizing compound (chlorine stabilizer) 6, said isocyanic acid 6 is added to said water 3, stabilizing said chlorine disinfectant 5, i.e. improving the UV stability of said chlorine 5.

Therefore, chlorinated isocyanuric acid 5, 6, as exemplary solid chloroisocyanurate (tablets) 5, 6, is added to said water 3 with said chlorine 5 and said isocyanic acid 6 then being dissolved in said water 3 while forming a chemical equilibrium between free chlorine and the chlorinated isocyanurate species.

As accumulated isocyanic acid 6 endanger disinfection safety and water quality an adequate (online) measurement/monitoring 140 of the concentration/level of said isocyanic acid 6 must be provided facilitating an efficient and effective water treatment/disinfection control 1, 100.

The measurement principle for said online measurement/monitoring of said isocyanic acid 6, 140 contained in said water 3 based on the photolytic chlorine degradation 8—with the chlorine concentration of said water 3 will be (online) measured in front of/before (initial chlorine concentration, 12, 110) and past/after (remaining chlorine concentration, 13, 130) a defined UV irradiation 7 of said water 3 intermediate/between 130.

The photolytic degradation 8 of said chlorine 5, i.e. said (online) measured change in concentration (Δchlorine), is first order decay to said isocyanic acid 6 contained in said water 3. Said online monitoring/measurement 140 of the isocyanic acid 6 will be facilitated by measuring 110, 130 said initial and remaining chlorine concentration 12, 13 and processing the isocyanic acid 6 (concentration) consequentially.

Based on that online monitoring 140 of the isocyanic acid 6 the water treatment control 1, 100 will be executed by controlling a dosing (not shown) of the chlorine 5 and the isocyanic acid 6 to said water 3—therefore controlling water disinfection and water quality.

The water 3—containing the chlorine disinfectant 5 and the chlorine stabilizer 6, i.e. containing the chlorine 5 and the isocyanic acid 6—enters as shown in FIG. 1 the “by pass” 2.

The “by pass” 2 comprises three sections 12, 13, 7 with a first section 12, an initial chlorine concentration measurement section 12, a second section 7, an UV irradiating section 7, and a third section 13, a remaining chlorine concentration measurement section 13—with said three sections 12, 13, 7 arranged in a flow direction 11 of said water 3 flowing through the “by pass” 2 by a constant flow rate, exemplary in a rage of about 5 L/h-35 L/h.

The initial chlorine concentration measurement section 12 as well as the remaining chlorine concentration measurement section 13 comprise a first 14 and second 15 online sensor 14, 15 being arranged at a first 16 and second 17 measuring point 16, 17 within the initial/remaining chlorine concentration measurement section 12, 13 for measuring 110, 130 a initial/remaining concentration of the free chlorine in the water 3—with the water 3 be irradiated 120 within the UV irradiating section 7 arranged between the concentration measurement sections 12, 13.

The first sensor 14 as well as the second sensor 15 are connected to an analyser and controller system 18, cited as a controller 18, via a circuit 19 controlling the concentration measurements of the chlorine—and—further—processing the measured (initial/remaining) chlorine concentration facilitating the water treatment control 1, 100.

The water 3—leaving the first section 12—enters the second section 7, i.e. a reaction chamber 9 with tubular low pressure UV lamps 20 inside, to be irradiated 120 with a defined UV irradiation 7 while flowing through the reaction chamber 9.

The reaction chamber 9 can have varying shape and size. FIG. 1 shows said reaction chamber 9 shaped as a cylinder being passed by the water 3 with said constant flow rate.

While the water 3 being irradiated 120 with UV a photolytic chlorine degradation 8 will be processed within the water 3 degrading the chlorine 5 contained in the water 3.

FIG. 1 shows an UV sensor 21 and an UV filter 22 being arranged at the UV lamps 20 used for controlling the irradiance of said UV irradiation 7 while measuring said UV irradiation 7 filtered by said UV filter 22.

The UV sensor 21 as well as the UV lamps 20 is also connected to the controller 18 via a circuit 19—being controlled by the controller 18 facilitating said defined UV irradiation 7.

The irradiation 7 of the water 3—provided with an irradiation dose of about 3000 J/m²—yields said photolytic chlorine degradation 8 (Δchlorine).

The photolytic chlorine degradation 8 (Δchlorine) depends, belong other parameters, the (defined) irradiance 7 of the UV source/lamps 20 as well as the isocyanic acid 6 contained in the water 3 while a knowledge of said Δchlorine measured allows processing and monitoring the isocyanic acid concentration.

Leaving the third section 13 the water 3 will leave the “by pass” 2 and be discharged in the main pool water circulation 4. As other option the water can be discharged after the measurement in by pass.

The first sensor 14 and the second sensor 15 are measuring n110, 130 the initial/remaining concentration (12, 13) of the free chlorine in the water 8 at the first and second measuring point 16, 17 while the sensor signals and sensor data according to said measured concentrations are transferred—via the circuit 19—to the controller 18 for processing.

The first and second sensor 14, 15 are realized as membrane sensors, i.e. as a membrane sensor FC1 or TC1 of Wallace & Tiernan (Wallace & Tiernan, Siemens, Water Technologies, Produktinformation zu Membransensor FC1 and TC1).

While knowing the UV irradiating parameters of the UV irradiation 7, as the dose, reaction time and fluence, as well as the flow rate of the water 3 and the UV/isocyanic acid dose response curve which has be determined experimentally preliminary the isocyanic acid 6 will be processed and monitored 140 by said controller 18.

Based on that online processing/monitoring 140 of the isocyanic acid 6 the water treatment control 1, 100 will be executed by controlling a dosing of the chlorine 5 and the isocyanic acid 6, i.e. said chlorinated isocyanuric acid 5, 6, to said water 3—therefore controlling water disinfection and water quality.

Therefore, in case of an increase of the isocyanic acid 6—online monitored 140 on basis of the photolytic chlorine degradation 8, i.e. Δchlorine measured and processed—exceeding a threshold—the dosing of the isocyanic acid 6 to said water 3 will be adjusted, i.e. will be reduced, while additional chlorine could be additionally added by dosing means (not shown) controlled by the controller 18 to maintain sufficient chlorine water disinfection.

### REFERENCE LIST

1 arrangement for a water treatment/chlorine disinfection control
2 by pass, flow pass
3 swimming pool water
4 main water circulation of said swimming pool water circulating, main pool water circulation
5 chlorine disinfectant, chlorine
6 chlorine stabilizer, isocyanic acid
7 UV irradiation (section)
8 photolytic chlorine degradation
9 reaction chamber
10 UV source
11 flow direction
12 initial chlorine concentration measurement (section)
13 remaining chlorine concentration measurement (section)
14 first online sensor
15 second online sensor
16 first measuring point
17 second measuring point
18 controller, analyser and controller system
19 circuit
20 tubular low pressure UV lamp(-s)
21 UV sensor
22 UV filter
100 method for a water treatment/chlorine disinfection control
110 measuring a concentration of a chlorine disinfectant contained in water before irradiating said water with an UV irradiation (initial chlorine concentration)
120 irradiating water with UV irradiation
130 measuring a concentration of a chlorine disinfectant contained in water after irradiating of said water (remaining chlorine concentration)
140 determining a concentration of a chlorine stabilizer using a initial chlorine concentration and a remaining chlorine concentration, online measurement/monitoring of a chlorine stabilizer

What is claimed is:

1. A method for a water treatment control of water to be treated containing a chlorine disinfectant stabilized by a chlorine stabilizer comprising:
measuring an initial concentration of said chlorine disinfectant in said water,
after measuring the concentration of said chlorine disinfectant in said water, irradiating said water with a UV irradiation,
measuring a remaining concentration of said chlorine disinfectant contained in said water after said irradiating of said water, and
determining a concentration of said chlorine stabilizer based on the measured initial chlorine concentration and the measured remaining chlorine concentration for said water treatment control.

2. The method of claim 1, wherein said chlorine disinfectant comprises at least one of: chlorine/chlorine gas, chlorine dioxide, hypochloric acid, and hypo chlorite.

3. The method of claim 1, wherein said chlorine stabilizer comprises isocyanic acid.

4. The method of claim 1, wherein said water to be treated is swimming pool water.

5. The method of claim 1, wherein determining the concentration of said chlorine stabilizer comprises:
determining an increase or decrease from said initial chlorine concentration to said remaining chlorine concentration while determining said concentration of said chlorine stabilizer, and
determining a decrease or increase of said concentration of said chlorine stabilizer based on the determined increase or decrease from said initial chlorine concentration to said remaining chlorine concentration.

6. The method of claim 1, wherein:
said initial chlorine concentration and said remaining chlorine concentration are measured online, and
said determination of the concentration of said chlorine stabilizer is determined online.

7. The method of claim 6, wherein said water treatment control uses said online determined chlorine stabilizer concentration.

8. An arrangement for a water treatment control of water to be treated containing a chlorine disinfectant stabilized by a chlorine stabilizer, comprising:
a flow pass of water with a UV source arrangement arranged at said flow pass and configured to irradiate said water with a UV irradiation,
a first measuring means arranged at said flow pass upstream of said UV source arrangement and configured to measure an initial concentration of said chlorine disinfectant in said water before irradiating said water with said UV irradiation (initial chlorine concentration),
a second measuring means arranged at said flow pass downstream of said UV source arrangement for measuring said concentration of said chlorine disinfectant contained in said water after said irradiating of said water (remaining chlorine concentration), and
a controlling means for determining a concentration of said chlorine stabilizer using said initial chlorine concentration and said remaining chlorine concentration for said water treatment control.

9. The arrangement of claim 8, wherein said UV source arrangement comprises one or more tubular low pressure UV lamps, especially operating in a range of 20 Watt-100 Watt and/or with an irradiation dose of about 200 J/m²-4000 J/m².

10. The arrangement of claim 8, arranged at a “by pass” of a main water circulation, especially of a main pool water circulation.
